(19) United States
(12) Patent Application Publication
(10) Pub. No.: US 2014/0242104 A1
(43) Pub. Date: Aug. 28, 2014

(54) SELF-ASSEMBLING PEPTIDE NANOPARTICLES AS VACCINES AGAINST INFECTION WITH NOROVIRUS

(71) Applicant: ALPHA-O PEPTIDES AG, Riehen (CH)

(72) Inventors: Peter Burkhard, Mansfield Center, CT (US); Caroline Kulangara, Basel (CH)

(73) Assignee: ALPHA-O PEPTIDES AG, Riehen (CH)

(21) Appl. No.: 14/350,426

(22) PCT Filed: Oct. 5, 2012
(86) PCT No.: PCT/EP2012/069684

§ 371 Date: Apr. 8, 2014

(30) Foreign Application Priority Data
Oct. 12, 2011 (EP) 11184906.3

(51) Int. Cl. A61K 39/12; C07K 14/005

(52) U.S. Cl. CPC A61K 39/12; C07K 14/005
USPC 424/186.1; 530/350

(57) ABSTRACT

Self-assembling peptide nanoparticles (SAPN) incorporating T-cell epitopes and displaying the P domain of the norovirus protein VP1 are described. The nanoparticles of the invention consist of aggregates of a continuous peptide chain comprising two coiled coil oligomerization domains connected by a linker segment wherein one or both oligomerization domains incorporate T-cell epitopes within their peptide sequence. These nanoparticles are useful as vaccines and adjuvants for the prevention and treatment of norovirus infections.

### FIELD OF THE INVENTION

The present invention relates to self-assembling peptide nanoparticles incorporating norovirus capsid proteins and T-cell epitopes. Furthermore, the invention relates to the use of such nanoparticles for vaccination against infection with norovirus.

### BACKGROUND OF THE INVENTION

The “Norwalk-Like Virus” now generally known as norovirus, is probably the most important viral pathogen of epidemic acute gastroenteritis which occurs in developed as well as in developing countries. Noroviruses are icosahedral, single stranded, positive-sense RNA viruses and belong to the Caliciviridae family. Their capsids are composed of 180 copies of one single major capsid protein. In the past, the biological analysis of human noroviruses had been held back because the virus resisted to grow in cell cultures and there was a lack of suitable animal models for virus cultivation. The only source of the virus were human stool samples obtained from human volunteer studies and from outbreaks, but the concentration of the virus in stool was so minute that virus detection with standard electron microscopy was not feasible. The recombinant expression of norovirus capsid proteins by baculoviruses, which are double stranded DNA viruses, in insect cells, however, has made it possible to study the epidemiology, immunology and pathogenesis of noroviruses. The capsid proteins that form the viral capsid self-assemble into so-called virus-like particles (VLPs). These VLPs are antigenetically and morphologically indistinguishable from authentic viruses found in human feces, thus providing a useful tool for the study of receptor—virus interactions and also for the development of immunological assays.

The structure of this capsid protein has been determined by X-ray crystallography (Prasad

BVV et al., Science 286, 287-290, 1999) and can be described as having two main domains, the S and P domain. The icosahedral shell is largely formed by the S domain (the N-terminal 225 residues) while the P domain (residues 225 to the C-terminus) forms a dimeric protrusion emanating from the shell. Residues 50 to 225 of the N-terminal S domain fold into a classical eight-stranded anti-parallel β sandwich, a common fold seen in many viral capsid proteins. The P domain is made of two subdomains: The P1 subdomain consists of residues 226 to 278 and 406 to 520; while the P2 subdomain consists of residues 279 to 405. The P2 subdomain is a large insertion starting from residues 278 and ending at residue 406. The section of amino acids 285 to 380 in the P2 subdomain folds into a compact barrel-like architecture that consists of six β strands.

Within the P1 subdomain, residues 226 to 278 contain three short stretches of β strands, whereas the C-terminal 114 residues contain six β strands and a well-defined a helix. To form a T=3 icosahedral structure, the capsid protein has to adapt to three quasi-equivalent positions. In the modular structure of the capsid protein, the S domain is involved in the icosahedral contacts, whereas the P domain is exclusively involved in the dimeric contacts. The P domains of the A and B subunits interact across the quasi twofold axes to form the dimeric protrusions as seen in the cryomicroscopy reconstruction. Similarly, the P domains of the C subunits interact across the icosahedral twofold axes. The NB and C/C dimers are stabilized mainly by interactions between the side chains of the participating monomers with a total contact area of about 2000 Å².

In contrast to the S and P1 domains, the P2 domain has a high sequence variation and therefore is believed to be critical in immune recognition and receptor binding. It has been shown that isolated P domains with the hinge (but lacking the S domain) form dimers in vitro that maintain binding to HBGA receptors. Noroviruses have been found to recognize human histo-blood group antigens as receptors. Among the histo-blood group antigens, the most commonly encountered blood groups are ABO (ABH) and Lewis. The biosynthetic pathways used in forming antigens in the ABH, Lewis, P, and I blood group systems are interrelated. Histo-blood group antigens have been linked to infection by several bacterial and viral pathogens. This suggests that the histo-blood group antigens are a recognition target for pathogens and may facilitate entry into a cell that expresses or forms a receptor-ligand bond with the antigens. While the exact nature of such an interaction is not currently known, close association of a pathogen that would occur with antigen binding may play a role in anchoring the pathogen to the cell as an initial step in the infection process. Human histo-blood group antigens are complex carbohydrates linked to glycoproteins or glycolipids that are present on the red blood cells and mucosal epithelial cells or as free antigens in biological fluids, such as blood, saliva, intestinal contents, and milk. These antigens are synthesized by sequential additions of monosaccharides to the antigen precursors by several glycosyltransferases that are genetically controlled and known as the ABO, Lewis, and secretor gene families. The prototype norovirus, the Norwalk Virus strain, represents one of these identified binding patterns that binds to histo-blood group antigens of types A and O secretors but not of non-secretors. The other known binding patterns include strain VA387 that recognize A, B and O secretors, and MOH that binds to A and B secretors. Human volunteer studies have shown the linkage of norovirus binding to HBGA with clinical infection, which demonstrated, for example, that individuals who are non-secretors were naturally resistant to NV infection following the challenge. Thus, it seems logical to expect that each of the other binding patterns has its own host ranges defined by blood types, although direct evidence for this hypothesis remains to be established. However, a recent human volunteer study using Snow Mountain Virus, a genotype II (GII of the three known genotypes) norovirus strain, did not reveal a clear correlation between infection and blood types, suggesting that factors other than histo-blood group antigens may play a role for the infection of this strain.

In light of the foregoing, it would be advantageous to provide a vaccine against norovirus that includes an immunogenic response to the P domain of the norovirus capsid.

The P domain on its own (i.e. lacking the N-terminal S domain) has been shown to form particles on its own, so-called P particles (Tan M, et al., Virology, 382, 115-132, 2008). They are being used to develop vaccines against norovirus infection and they are also used to display other pathogen related peptides and proteins to engineer a multi-component vaccine (Tan M, et al., J. Virol, 85, 753-764, 2011).

Peptide nanoparticles are described in WO 2004/071493. Peptide nanoparticles incorporating T-cell epitopes are described in WO 2009/109428.

### SUMMARY OF THE INVENTION

The invention relates to a self-assembling peptide nanoparticle (SAPN) consisting of aggregates of a multitude of building blocks of formula (I) consisting of a continuous chain comprising a peptide oligomerization domain D1, a linker segment L, a peptide oligomerization domain D2, and the P domain or the P2 subdomain of norovirus protein VP1

D1-L-D2-P (I),

wherein D1 is a coiled coil peptide that forms oligomers (D1)ₘ of m subunits D1, D2 is a coiled coil peptide that forms dimers (D2)₂ of 2 subunits D2, m is either 3 or 5, L is a bond or a short linker segment, either D1 or D2 or both D1 and D2 incorporate one or more T-cell epitopes within the oligomerization domain, and wherein D2 is substituted by P representing the P domain or the P2 subdomain of the norovirus VP1 protein.

### BRIEF DESCRIPTION OF THE FIGURES

FIG. 1: Vector map of pPEP-T. The insertion sites used for sub-cloning are shown in larger letters. For the external insertion sites NcoI and EcoRI the nucleotide numbers of the original vector are indicated. Origin, ampilicilin resistance with promoter and T7 promoter and terminator are shown. Restriction sites for subcloning (NcoI and EcoRI) are indicated with by number.

FIG. 2: Expression of the SAPN construct comprising the sequence shown in SEQ ID NO:1 in E coli BL21 (DE3) cells by SDS-PAGE.

ui=uninduced, i=induced. In the lane i, the strong band is assigned to the construct comprising SEQ ID NO:1, Noro-SAPN, with a molecular weight of 44.3 kDa. First and last lanes: Molecular weight (kDa) markers; other lanes: other uninduced batches.

FIG. 3: Analysis of purification of Noro-SAPN on a nickel affinity column by a stepwise pH gradient from pH 8.0 to pH 4.5, followed by high concentration of imidazole by SDS-PAGE.

FT=Flow-through; M=monomer; D=Degradation; IE=imidazole elution at 250 mM imidazole, pH 8.0.

FIG. 4: Electron micrographs of Noro-SAPN refolded in pH 6.8, 80 mM NaCl, 20 mM MES, 5% glycerol. A, B, C are different enlargements (see bar).

### DETAILED DESCRIPTION OF THE INVENTION

The invention relates to a self-assembling peptide nanoparticle (SAPN) consisting of aggregates of a multitude of building blocks of formula (I) consisting of a continuous chain comprising a peptide oligomerization domain D1, a linker segment L, a peptide oligomerization domain D2, and the P domain or the P2 subdomain of norovirus protein VP1

D1-L-D2-P (I),

wherein D1 is a coiled coil peptide that forms oligomers (D1)ₘ of m subunits D1, D2 is a coiled coil peptide that forms dimers (D2)₂ of 2 subunits D2, m is either 3 or 5, L is a bond or a short linker segment, either D1 or D2 or both D1 and D2 incorporate one or more T-cell epitopes within the oligomerization domain, and wherein D2 is substituted by P representing the P domain or the P2 subdomain of the norovirus VP1 protein.

In the continuous chain D1-L-D2-P, peptide oligomerization domain D1 may further be substituted by peptides at its N-terminal end, for example by peptides consisting of 1 to 100 amino acids, in particular 1 to 20 amino acids. One particular example is a His-tag sequence for purification purposes.

In the continuous chain D1-L-D2-P, the peptide oligomerization domain D2 and the P domain or the P2 subdomain of the norovirus VP1 protein may be connected directly by a bond, or in the alternative, by a peptide chain of 1 to 20, preferably 1 to 10 amino acids. At its C-terminal end, the P domain or the P2 subdomain may be further substituted by peptides, for example by peptides consisting of 1 to 100 amino acids, in particular 1 to 20 amino acids.

A peptide (or polypeptide) is a chain or sequence of amino acids covalently linked by amide bonds. The peptide may be natural, modified natural, partially synthetic or fully synthetic. Modified natural, partially synthetic or fully synthetic is understood as meaning not occurring in nature. The term amino acid embraces both naturally occurring amino acids selected from the 20 essential natural α-L-amino acids, synthetic amino acids, such as α-D-amino acids, 6-aminohexanoic acid, norleucine, homocysteine, or the like, as well as naturally occurring amino acids which have been modified in some way to alter certain properties such as charge, such as phoshoserine or phosphotyrosine, or the like. The term amino acid further embraces derivatives of amino acids wherein the amino group forming the amide bond is alkylated, or a side chain amino, hydroxy or thio function is alkylated or acylated, or a side chain carboxy function is amidated or esterified.

Preferred are peptides consisting of naturally occurring amino acids and derivatives of such amino acids wherein the amino group forming the amide bond is alkylated, or a side chain amino, hydroxy or thio function is alkylated or acylated, or a side chain carboxy function is amidated or esterified.

A short linker segment L is preferably a peptide chain, e.g. a peptide chain consisting of 1 to 20 amino acids, in particular 1 to 6 amino acids.

A coiled coil is a peptide sequence with a contiguous pattern of mainly hydrophobic residues spaced 3 and 4 residues apart, which assembles to form a multimeric bundle of helices, as will be explained in more detail hereinbelow.

“A coiled coil that incorporates T-cell epitopes” means that the corresponding epitope is comprised within an oligomerization domain such that the coiled coil amino acid sequences flanking the epitope at the N-terminal and the C-terminal ends force the epitope to adapt a conformation which is still a coiled coil in line with the oligomerization properties of the oligomerization domain comprising the epitope. In particular, “incorporated” excludes a case wherein the epitope is attached at either end of the coiled coil oligomerization domain.

In the context of this document the term T-cell epitope shall be used to refer to both CTL and HTL epitopes.

D1, D2 and P may optionally be further substituted by targeting entities, or substituents reinforcing the adjuvant properties of the nanoparticle, such as an immunostimulatory nucleic acid, preferably an oligodeoxynucleotide containing deoxyinosine, an oligodeoxynucleotide containing deoxyuridine, an oligodeoxynucleotide containing a CG motif, or an inosine and cytidine containing nucleic acid molecule. Other substituents reinforcing the adjuvant properties of the nanoparticle are antimicrobial peptides, such as cationic peptides, which are a class of immunostimulatory, positively charged molecules that are able to facilitate and/or improve adaptive immune responses. An example of such a peptide with immunopotentiating properties is the positively charged artificial antimicrobial peptide KLKLLLLLKLK (SEQ ID NO:2) which induces potent protein-specific type-2 driven adaptive immunity after prime-boost immunizations.

Optional substituents, e.g. those optional substituents described in the preceding paragraph, are preferably connected to suitable amino acids close to the N-terminal end of the oligomerization domain D1 or to a further peptide substituted at the N-terminal end of the oligomerization domain D1. On self-assembly of the peptide nanoparticle, such substituents will then be presented at the surface of the SAPN.

In a most preferred embodiment the substituent is another peptide sequence 51 representing a simple extension of the peptide chain D1-L-D2-P at the N-terminal end of D1 to generate a combined single peptide sequence, which may be expressed in a recombinant protein expression system as one single molecule.

A peptide oligomerization domain is a peptide that has a tendency to form oligomers by association or aggregation through hydrophobic, hydrophilic or ionic interactions, in particular hydrogen bonding. For example, a peptide dimerization domain D is a peptide which forms dimers D₂ in solution, usually under physiological conditions. A peptide trimerization domain D forms trimers D₃, a tetramerization domain D forms tetramers D₄, and a pentamerization domain D forms pentamers D₅ in solution. A tendency to form oligomers means that such peptides can form oligomers depending on the conditions, e.g. under denaturing conditions they are monomers, while under physiological conditions they may form corresponding oligomers. Under predefined conditions they adopt one single oligomerization state, which is needed for nanoparticle formation. However, their oligomerization state may be changed upon changing conditions, e.g. from dimers to trimers upon increasing salt concentration (Burkhard P. et al., Protein Science 2000, 9:2294-2301) or from pentamers to monomers upon decreasing pH.

Peptide oligomerization domains are well-known (Burkhard P. et al., Trends Cell Biol 2001, 11:82-88). In the present invention the oligomerization domains D1 and D2 are coiled coil domains. A coiled coil is a peptide sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled coils with sequences including some irregular distribution of the 3 and 4 residues spacing are also contemplated. Hydrophobic residues are in particular the hydrophobic amino acids Val, Ile, Leu, Met, Tyr, Phe and Trp. Mainly hydrophobic means that at least 50% of the residues must be selected from the mentioned hydrophobic amino acids.

For example, in a preferred monomeric building block of formula (I), D1 and D2 are peptides of any of the formulae

[aa(a)-aa(b)-aa(c)-aa(d)-aa(e)-aa(f)-aa(g)]ₓ (IIa),

[aa(b)-aa(c)-aa(d)-aa(e)-aa(f)-aa(g)-aa(a)]ₓ (IIb),

[aa(c)-aa(d)-aa(e)-aa(f)-aa(g)-aa(a)-aa(b)]ₓ (IIc),

[aa(d)-aa(e)-aa(f)-aa(g)-aa(a)-aa(b)-aa(d)]ₓ (IId),

[aa(e)-aa(f)-aa(g)-aa(a)-aa(b)-aa(c)-aa(d)]ₓ (IIe),

[aa(f)-aa(g)-aa(a)-aa(b)-aa(c)-aa(d)-aa(e)]ₓ (IIf),

[aa(g)-aa(a)-aa(b)-aa(c)-aa(d)-aa(e)-aa(f)]ₓ (IIg),

wherein aa means an amino acid or a derivative thereof, aa(a), aa(b), aa(c), aa(d), aa(e), aa(f), and aa(g) are the same or different amino acids or derivatives thereof, preferably aa(a) and aa(d) are the same or different hydrophobic amino acids or derivatives thereof; and X is a figure between 2 and 20, preferably between 3 and 10, in particular 3, 4, 5, or 6.

Hydrophobic amino acids are Val, Ile, Leu, Met, Tyr, Phe and Trp.

A heptad is a heptapeptide of the formula aa(a)-aa(b)-aa(c)-aa(d)-aa(e)-aa(f)-aa(g) (IIa) or any of its permutations of formulae (IIb) to (IIg).

Preferred are monomeric building blocks of formula (I) wherein both peptide oligomerization domains D1 and D2 are

(1) a peptide of any of the formulae (IIa) to (IIg) wherein X is 3, and aa(a) and aa(d) are selected from the 20 natural α-L-amino acids such that the sum of scores from Table 1 for these 6 amino acids is at least 14, and such peptides comprising up to 17 further heptads; or

(2) a peptide of any of the formulae (IIa) to (IIg) wherein X is 3, and aa(a) and aa(d) are selected from the 20 natural α-L-amino acids such that the sum of scores from Table 1 for these 6 amino acids is at least 12, with the proviso that one amino acid aa(a) is a charged amino acid able to form an inter-helical salt bridge to an amino acid aa(d) or aa(g) of a neighboring heptad, or that one amino acid aa(d) is a charged amino acid able to form an inter-helical salt bridge to an amino acid aa(a) or aa(e) of a neighboring heptad, and such peptides comprising up to two further heptads. A charged amino acid able to form an inter-helical salt bridge to an amino acid of a neighboring heptad is, for example, Asp or Glu if the other amino acid is Lys, Arg or His, or vice versa.

TABLE 1
Scores of amino acid for determination of preference

| Amino acid | Position aa(a) | Position aa(d) |
|---|---|---|
| L (Leu) | 3.5 | 3.8 |
| M (Met) | 3.4 | 3.2 |
| I (Ile) | 3.9 | 3.0 |
| Y (Tyr) | 2.1 | 1.4 |
| F (Phe) | 3.0 | 1.2 |
| V (Val) | 4.1 | 1.1 |
| Q (Gln) | −0.1 | 0.5 |
| A (Ala) | 0.0 | 0.0 |
| W (Trp) | 0.8 | −0.1 |
| N (Asn) | 0.9 | −0.6 |
| H (His) | −1.2 | −0.8 |
| T (Thr) | 0.2 | −1.2 |
| K (Lys) | −0.4 | −1.8 |
| S (Ser) | −1.3 | −1.8 |
| D (Asp) | −2.5 | −1.8 |
| E (Glu) | −2.0 | −2.7 |
| R (Arg) | −0.8 | −2.9 |
| G (Gly) | −2.5 | −3.6 |
| P (Pro) | −3.0 | −3.0 |
| C (Cys) | 0.2 | −1.2 |

Also preferred are monomeric building blocks of formula (I) wherein one or both peptide oligomerization domains D1 or D2 are selected from the following preferred peptides:

(11) Peptide of any of the formulae (IIa) to (IIg) wherein
aa(a) is selected from Val, Ile, Leu and Met, and a derivative thereof, and
aa(d) is selected from Leu, Met, Val and Ile, and a derivative thereof.

(12) Peptide of any of the formulae (IIa) to (IIg) wherein one aa(a) is Asn and the other aa(a) are selected from Asn, Ile and Leu, and aa(d) is Leu. Such a peptide is usually a dimerization domain as present in D2.

(13) Peptide of any of the formulae (IIa) to (IIg) wherein aa(a) and aa(d) are both Ile. Such a peptide is usually a trimerization domain (m=3) as present in D1.

(14) Peptide of any of the formulae (IIa) to (IIg) wherein aa(a) and aa(d) are both Trp. Such a peptide is usually a pentamerization domain (m=5) as present in D1.

(15) Peptide of any of the formulae (IIa) to (IIg) wherein aa(a) and aa(d) are both Phe. Such a peptide is usually a tetramerization domain (m=4) as present in D1.

(16) Peptide of any of the formulae (IIa) to (IIg) wherein aa(a) and aa(d) are both either Trp or Phe. Such a peptide is usually a pentamerization domain (m=5) as present in D1.

(17) Peptide of any of the formulae (IIa) to (IIg) wherein aa(a) is either Leu or Ile, and one aa(d) is Gln and the other aa(d) are selected from Gln, Leu and Met. Such a peptide has the potential to be a pentamerization domain (m=5) as present in D1.

Other preferred peptides are peptides (1), (2), (11), (12), (13), (14), (15), (16) and (17) as defined hereinbefore, and wherein further

(21) at least one aa(g) is selected from Asp and Glu and aa(e) in a following heptad is Lys, Arg or His; and/or

(22) at least one aa(g) is selected from Lys, Arg and His, and aa(e) in a following heptad is Asp or Glu, and/or

(23) at least one aa(a to g) is selected from Lys, Arg and His, and an aa(a to g) 3 or 4 amino acids apart in the sequence is Asp or Glu. Such pairs of amino acids aa(a to g) are, for example aa(b) and aa(e) or aa(f).

Coiled coil prediction programs such as COILS

(http://www.ch.embnet.org/software/COILS_form.html; Gruber M. et al., J. Struct. Biol. 2006, 155(2):140-5) or MULTICOIL
(http://groups.csail.mit.edu/cb/multicoil/cgi-bin/multicoil.cgi) can predict coiled coil forming peptide sequences. Therefore, in a monomeric building block of formula (I), D1 and D2 are peptides that contain at least a sequence two heptad-repeats long that is predicted by the coiled coil prediction program COILS to form a coiled coil with higher probability than 0.9 for all its amino acids with at least one of the window sizes of 14, 21, or 28.

In a more preferred monomeric building block of formula (I), D1 and D2 are peptides that contain at least one sequence three heptad-repeats long that is predicted by the coiled coil prediction program COILS to form a coiled coil with higher probability than 0.9 for all its amino acids with at least one of the window sizes of 14, 21, or 28.

In another more preferred monomeric building block of formula (I), D1 and D2 are peptides that contain at least two separate sequences two heptad-repeats long that are predicted by the coiled coil prediction program COILS to form a coiled coil with higher probability than 0.9 for all its amino acids with at least one of the window sizes of 14, 21, or 28.

Most preferred are the coiled coil sequences and monomeric building blocks described in the examples.

A building block architecture according to formula (I) is clearly distinct from viral capsid proteins. Viral capsids are composed of either one single protein, which forms oligomers of 60 or a multiple thereof, as e.g. the hepatitis virus B particles (EP 1 262 555, EP 0 201 416), or of more than one protein, which co-assemble to form the viral capsid structure, which can adopt also other geometries apart from icosahedra, depending on the type of virus (Fender P. et al., Nature Biotechnology 1997, 15:52-56). Self-assembling peptide nanoparticles (SAPN) of the present invention are also clearly distinct from virus-like particles, as they (a) are constructed from other than viral capsid proteins and (b) that the cavity in the middle of the nanoparticle is too small to accommodate the DNA/RNA of a whole viral genome.

### Self-Assembling Peptide Nanoparticles: LCM Units

Self-assembling peptide nanoparticles (SAPN) are formed from monomeric building blocks of formula (I). If such building blocks assemble, they will form so-called “LCM units”. The number of monomeric building blocks, which will assemble into such an LCM unit will be defined by the least common multiple (LCM). Hence, if for example the oligomerization domains of the monomeric building block form a pentamer (D1)₅ (m=5) and a dimer (D2)₂ (n=2), 10 monomers will form an LCM unit. If the linker segment L has the appropriate length, this LCM unit may assemble in the form of a spherical peptide nanoparticle.

Self-assembling peptide nanoparticles (SAPN) may be formed by the assembly of only one or more than one LCM units (Table 2). Such SAPN represent topologically closed structures.

TABLE 2
Possible combinations of oligomerization states

| | | | Polyhedron | | No. of | No. of Building |
|---|---|---|---|---|---|---|
| ID No. | m | n | Type | LCM | LCM Units | Blocks |
| 1 | 5 | 2 | dodecahedron/ | 10 | 6 | 60 |
| | | | icosahedrons | | | |
| 2 | 3 | 2 | tetrahedron | 6 | 2 | 12 |

### Regular Polyhedra

There exist five regular polyhedra, the tetrahedron, the cube, the octahedron, the dodecahedron and the icosahedron. They have different internal rotational symmetry elements. The tetrahedron has a 2-fold and two 3-fold axes, the cube and the octahedron have a 2-fold, a 3-fold and a 4-fold rotational symmetry axis, and the dodecahedron and the icosahedron have a 2-fold, a 3-fold and a 5-fold rotational symmetry axis. In the cube the spatial orientation of these axes is exactly the same as in the octahedron, and also in the dodecahedron and the icosahedron the spatial orientation of these axes relative to each other is exactly the same. Hence, for the purpose of SAPN of the invention the dodecahedron and the icosahedron can be considered to be identical. The dodecahedron/icosahedron is built up from 60 identical three-dimensional building blocks (Table 2). These building blocks are the asymmetric units (AUs) of the polyhedron. They are tri-pyramids and each of the pyramid edges corresponds to one of the rotational symmetry axes, hence these AUs will carry at their edges 2-fold, 3-fold, and 5-fold symmetry elements. If these symmetry elements are generated from peptide oligomerization domains such AUs are constructed from monomeric building blocks as described above. It is sufficient to align the two oligomerization domains D1 and D2 along two of the symmetry axes of the AU. If these two oligomerization domains form stable oligomers, the symmetry interface along the third symmetry axis will be generated automatically, and it may be stabilized by optimizing interactions along this interface, e.g. hydrophobic, hydrophilic or ionic interactions, or covalent bonds such as disulfide bridges.

Assembly to Self-Assembling Peptide Nanoparticles (SAPN) with Regular Polyhedral Symmetry

To generate self-assembling peptide nanoparticles (SAPN) with a regular geometry (dodecahedron, icosahedron), more than one LCM unit is needed. E.g. to form a icosahedron from a monomer containing dimeric and pentameric oligomerization domains, 6 LCM units, each composed of 10 monomeric building blocks are needed, i.e. the peptide nanoparticle with regular geometry will be composed of 60 monomeric building blocks. The combinations of the oligomerization states of the two oligomerization domains needed and the number of LCM units to form the two possible polyhedra are listed in Table 2.

Whether the LCM units will further assemble to form regular polyhedra composed of more than one LCM unit depends on the geometrical alignment of the two oligomerizations domains D1 and D2 with respect to each other, especially on the angle between the rotational symmetry axes of the two oligomerization domains. This is governed by i) the interactions at the interface between neighboring domains in a nanoparticle, ii) the length of the linker segment L, iii) the shape of the individual oligomerization domains. This angle is larger in the LCM units compared to the arrangement in a regular polyhedron. Also this angle is not identical in LCM units as opposed to the regular polyhedron. If this angle is restricted to the smaller values of the regular polyhedron (by means of hydrophobic, hydrophilic or ionic interactions, or a covalent disulfide bridge) and the linker segment L is short enough, a given number of topologically closed LCM units each containing a defined number of monomeric building blocks will then further anneal to form a regular polyhedron (Table 2), or enclose more monomeric building blocks to from nanoparticles lacking the strict internal symmetry of a polyhedron.

If the angle between the two oligomerization domains is sufficiently small (even smaller than in a regular polyhedron with icosahedral symmetry), then a large number (several hundred) peptide chains can assemble into a peptide nanoparticle. If the angle between the two helices is smaller, consequently more than 60 peptide chains can assemble into a SAPN. In such a design the SAPNs may have a molecular weight corresponding to several times 60 peptide chains as described by the theory of quasi-equivalence and the tiling theory of viral capsids for “all-pentamer” virus architectures.

### T-Cell Epitopes

Since the T-cell epitopes—as opposed to the B-cell epitopes—do not need to be displayed on the surface of a carrier to stimulate the immune system, they can be incorporated into the core scaffold of the SAPN, i.e. the coiled coil sequence of an oligomerization domain. In the present invention it is shown how the features of MHC binding of T-cell epitopes, which requires an extended conformation for MHC binding, can be combined with the features of coiled coil formation, which requires α-helical conformation for coiled coil formation, such that these epitopes can be both, part of the coiled coil scaffold of the SAPN as well as being able to bind to the respective MHC molecules. It should be noted that not all coiled coil sequences will be able to bind to MHC molecules and not all T-cell epitopes can be incorporated into a coiled coil structure.

### Sources of T-Cell Epitopes

To incorporate T-cell epitopes into an oligomerization domain leading finally to a self-assembling peptide nanoparticle (SAPN), the T-cell epitopes can be chosen from different sources: For example, the T-cell epitopes can be determined by experimental methods, they are known from literature, they can be predicted by prediction algorithms based on existing protein sequences of a particular pathogen, or they may be de novo designed peptides or a combination of them.

There is a wealth of known T-cell epitopes available in the scientific literature. These T-cell epitopes can be selected from a particular pathogen, or they may be de novo designed peptides with a particular feature, e.g. the PADRE peptide (U.S. Pat. No. 5,736,142) that binds to many different MHC II molecules, which makes it a so-called promiscuous T-cell epitope. There exist commonly accessible databases that contain thousands of different T-cell epitopes, for example the MHC-database “MHCBN VERSION 4.0” (http://www.imtech.res.in/raghava/mhcbn/index.html) or the Immune Epitope Database IEDB (http://www.iedb.org/) or others.

It is well known and well documented that incorporation of HTL epitopes into an otherwise not immunogenic peptide sequence or attaching it to a non-peptidic antigen can make those much more immunogenic. The PanDR binding peptide HTL epitope PADRE has widely been used in vaccine design for a malaria, Alzheimer and many others vaccines.

According to the definition of the MHCBN database (supra) T-cell epitopes are peptides that have binding affinities (10₅₀ values) of less than 50,000 nM to the corresponding MHC molecule. Such peptides are considered as MHC binders. According to this definition, as of August 2006, in the Version 4.0 of the MHCBN database the following data is available: 20717 MHC binders and 4022 MHC non-binders.

Suitable T-cell epitopes can also be obtained by using prediction algorithms. These prediction algorithms can either scan an existing protein sequence from a pathogen for putative T-cell epitopes, or they can predict, whether de novo designed peptides bind to a particular MHC molecule. Many such prediction algorithms are commonly accessible on the internet. Examples are SVRMHCdb (http://svrmhc.umn.edu/SVRMHCdb; J. Wan et al., BMC Bioinformatics 2006, 7:463), SYFPEITHI (http://www.syfpeithi.de), MHCPred

(http://www.jenner.ac.uk/MHCPred), motif scanner
(http://hcv.lanl.gov/content/immuno/motif_scan/motif_scan) or NetMHCIIpan
(http://www.cbs.dtu.dk/services/NetMHCIIpan) for MHC II binding molecules and
NetMHCpan (http://www.cbs.dtu.dk/services/NetMHCpan) for MHC I binding epitopes.

HTL epitopes as described herein and preferred for the design are peptide sequences that are either measured by biophysical methods or predicted by NetMHCIIpan to bind to any of the MHC II molecules with binding affinities (IC₅₀ values) better than 500 nM. These are considered weak binders. Preferentially these epitopes are measured by biophysical methods or predicted by NetMHCIIpan to bind to the MHC II molecules with IC₅₀ values better than 50 nM. These are considered strong binders.

CTL epitopes as described herein and preferred for the design are peptide sequences that are either measured by biophysical methods or predicted by NetMHCpan to bind to any of the MHC I molecules with binding affinities (IC₅₀ values) better than 500 nM. These are considered weak binders. Preferentially these epitopes are measured by biophysical methods or predicted by NetMHCpan to bind to the MHC I molecules with IC₅₀ values better than 50 nM. These are considered strong binders.

### Places for T-Cell Epitopes

The T-cell epitopes can be incorporated at several places within the peptide sequence of the coiled coil oligomerization domains D1 and/or D2. To achieve this, the particular sequence with the T-cell epitope has to obey the rules for coiled coil formation as well as the rules for MHC binding. The rules for coiled coil formation have been outlined in detail above. The rules for binding to MHC molecules are incorporated into the MHC binding prediction programs that use sophisticated algorithms to predict MHC binding peptides.

There are many different HLA molecules, each of them having a selection of amino acids in their sequence that will best bind to it. For example, corresponding binding motifs are summarized in Tables 3 and 4 of WO 2009/109428.

Engineering T-Cell Epitopes into Coiled Coils

To engineer SAPN that incorporate T-cell epitopes in the coiled coil oligomerization domain of the SAPN, three steps have to be taken. In a first step a candidate T-cell epitope has to be chosen by using known T-cell epitopes from the literature or from databases or predicted T-cell epitopes by using a suitable epitope prediction program. In a second step a proteasomal cleavage site has to be inserted at the C-terminal end of the CTL epitopes. This can be done by using the prediction program for proteasomal cleavage sites PAProc (http://www.paproc2.de/paproc1/paproc1.html; Hadeler K. P. et al., Math. Biosci. 2004, 188:63-79) and modifying the residues immediately following the desired cleavage site. This second step is not required for HTL epitopes. In the third and most important step the sequence of the T-cell epitope has to be aligned with the coiled coil sequence such that it is best compatible with the rules for coiled coil formation as outlined above. Whether the sequence with the incorporated T-cell epitope will indeed form a coiled coil can be predicted, and the best alignment between the sequence of the T-cell epitope and the sequence of the coiled coil repeat can be optimized by using coiled coil prediction programs such as COILS

(http://www.ch.embnet.org/software/COILS_form.html; Gruber M. et al., J. Struct. Biol. 2006, 155(2):140-5) or MULTICOIL
(http://groups.csail.mit.edu/cb/multicoil/cgi-bin/multicoil.cgi), which are available on the internet.

Even if it is not possible to find a suitable alignment—maybe because the T-cell epitope contains a glycine or even a proline which is not compatible with a coiled coil structure—the T-cell epitope may be incorporated into the oligomerization domain. In this case the T-cell epitope has to be flanked by strong coiled coil forming sequences of the same oligomerization state. This will either stabilize the coiled coil structure to a sufficient extent or alternatively it can generate a loop structure within this coiled coil oligomerization domain.

### Preferred Design

To engineer a SAPN with the best immunological profile for a given particular application the following considerations have to be taken into account:

CTL epitopes require a proteasomal cleavage site at their C-terminal end. The epitopes should not be similar to human sequences to avoid autoimmune responses. Accordingly a SAPN is preferred wherein at least one of the T-cell epitopes is a norovirus-specific CTL epitope, and, in particular, wherein the sequence further contains a proteasomal cleavage site after the CTL epitope.

Likewise preferred is a SAPN wherein at least one of the T-cell epitopes is a HTL epitope, in particular, a pan-DR-binding HTL epitope. Such pan-DR-binding HTL epitopes bind to many of the MHC class II molecules and are therefore recognized in a majority of healthy individuals, which is critical for a good vaccine.

Also preferred is a SAPN wherein the sequence D1-L-D2 contains a series of overlapping T-cell epitopes.

A norovirus vaccine preferably contains both HTL and CTL epitopes. The HTL epitopes should be as promiscuous as possible. They do not necessarily need to be derived from the pathogen but can be peptides that elicit a strong T-help immune response. An example would be the PADRE peptide. Preferably these are the T-cell epitopes that are incorporated into the D1-L-D2 core sequence of the SAPN. The CTL epitopes need to be norovirus-specific, they need to have C-terminal proteasomal cleavage sites.

The SAPN of the present invention has the following aspects which make it unique when compared to the known nanoparticles:

The design of a dimeric coiled coil with T-cell epitopes incorporated into it leads to a SAPN to which the P domain or the P2 subdomain of the norovirus VP 1 protein can be attached.

The fragment of the P subdomain is such that it can be attached to the SAPN and still from nanoparticles. This is not trivial as this corresponds to about 300 additional amino acids (compared to the 100 amino acids forming the nanoparticle core).

In particular, successful attachment of the P subdomain to the SAPN nanoparticle is not trivial and successful SAPN nanoparticle formation cannot readily be expected for the following reasons:
1. The P protein in itself is a highly flexible protein chararcterized by a large degree of structural mobility in the loops of the surface of the P2 subdomain that can switch between open and closed conformations. (Taube S et al., J Virol. 84(11), 5695-705, 2010).
2. The P protein most likely undergoes a so-called viral maturation process that involves major conformational changes as evidenced by X-ray crystal structures that show lifting off by 16 Å and rotation of the protein by 40 degrees between different maturation stages of the viral capsid. (Katpally U et al., J Virol. 82(5), 2079-88, 2008). This makes design of P2-containing SAPN a challenging protein design task.
3. The P2 subdomain in solution forms octameric nanoparticles (Tan M et al., Virology, 382(1), 115-23, 2008). Attachement of this P2 subdomain as octameric nanoparticles to the SAPN would obviously not work and hence the P2 subdomain would be expected to interfere with proper SAPN nanoparticle formation. This is even more so as the P particle in itself is not homogeneous but rather forms different aggregations states of 12-mer, 18-mer, 24-mer and 36-mer complexes (Bereszczak J Z et al., J Struct Biol. 177(2), 273-82, 2012).
4. Finally, modification of the protein sequence of the P2 subdomain leads to novel aggregates depending on the type of modification (Tan Metal., Virology, 382(1), 115-23, 2008). Modifications as small as insertion of a single amino acid, the addition of a flag tag or a change of the argininge cluster all alter the aggregation state of the P protein. Hence, a considerably larger modification such as the attachment of the whole SAPN sequence is expected to have a significant impact on the biophysical behavior of the P protein, and successful SAPN nanopaparticle formation therefore represents a major breakthrough in protein design.

As compared to norovirus VLPs as vaccines, the SAPN of the present invention allow for more flexibility in protein design for the optimization of the immune response as well as the biophysical properties of the vaccine. For example engineering of HTL epitopes into the backbone of the SAPN as described in WO 2009/109428 will make the SAPN of the present invention highly immunogenic. The biophysical stability of the SAPN of the invention can also be optimized by engineering optimal coiled coil sequences into the core of the SAPN. Along with this, the refolding properties of the SAPN of the invention can be optimized by adjusting the peptide sequence according to coiled coil folding principles as described herein below to allow for best refolding properties and optimal shelf-life of the SAPN. Such flexibility for protein engineering is not given for norovirus VLPs and hence best refolding properties and optimal shelf-life are much harder to accomplish with norovirus VLPs.

The same consideration make the SAPN of the invention superior to the so-called P particles, which are composed of the P domain only of norovirus. Especially the biophysical stability of these P particles is rather weak and is very difficult to control and optimize. Attempts have been taken with the engineering of cysteines into the terminus of the P particle sequence to increase the stability.

But also the immunogenicity of the SAPN of the present invention can be improved compared to the P particles by the same methods as outlined here within and in WO 2009/109428.

### Design of a Noro-SAPN

A particular example of a Noro-SAPN according to the invention is the following construct:

For ease of purification the Noro-SAPN starts with the sequence

(SEQ ID NO: 3)
MGHHHHHHASGS,

which contains a His-tag for nickel affinity purification and, at the DNA level, restriction sites for further sub-cloning.

For D1 a pentamerization domain was chosen (m=5). The particular pentameric coiled coil is a novel modification of the tryptophan-zipper pentamerization domain (Liu J et al., Proc Natl Acad Sci U S A 2004; 101(46):16156-61, RCSB Protein Data Bank pdb-entry 1T8Z). The original tryptophan-zipper pentamerization domain has the sequence

(SEQ ID NO: 4)
SSNAKWDQWSSDWQTWNAKWDQWSNDWNAWRSDWQAWKDDWARWNQRWD
NWAT.

The modified coiled coil sequence (SEQ ID NO:5) of the pentamerization domain used for noro-SAPN starts at position 13, ends at position 42 and contains 6 additional charges at the positions 14, 15, 22, 24, 29 and 32 and a valine instead of lysine at position 38. The valine allows the engineering of a restriction site Sal I into the sequence for further sub-cloning.

(SEQ ID NO: 5)
13-WEKWNAKWDEWKNDWNDWRRDWQAWVDDWA-42.

This sequence is extended by 8 amino acids derived from the helix-turn-helix motif of the channel-forming domain of colicin E1.

The original channel-forming domain of colicin E1 RCSB Protein Data Bank pdb-entry 2i88 from residue 25 to 41 has the sequence

(SEQ ID NO: 6)
25-YQTLTEKYGEKYSKMA-41

Into this helix-turn-helix motif the following modifications were introduced: At both position 26 and 30 a tryptophane residue replaces the original residues (glutamine and glutamate) and at both position 35 and 39 a leucine replaces the original residues (lysine and methionine). The first two modifications allow the whole sequence including the tryptophane zipper to form a pentameric coiled coil domain with the following sequence (SEQ ID NO:7)

(SEQ ID NO: 7)
WEKWNAKWDEWKNDWNDWRRDWQAWVDDWAYWTLTWK

The second two modifications allow the sequence to form a dimeric coiled coil when connected with the de novo designed dimeric coiled coil sequence of D2. This modified helix-turn-helix motif thus naturally connects the pentamer to the dimer, hence the linker L is formed by the two amino acids tyrosine-glycine, which corresponds to the amino acids No. 32 and 33 of SEQ ID NO:6, above.

This leads to D2 of the following sequence:

(SEQ ID NO: 8)
ELYSKLAELERRNEELERRLEELARFVAALSMRLAELERRNEELAR

For another closely related de novo sequence

(SEQ ID NO: 9)
ELYSKLAELERRLEELERRLEELARFVAALSMRLAELERRLEELAR

it has been shown that a dimer is formed even under completely denaturing conditions of an SDS-PAGE.

By engineering asparagine residues at few select aa(a) positions of the coiled coil the dimer formation can even more strongly be enforced in the structure. The asparagine residues at position 13 and 41 in SEQ ID NO:8 were engineered to assure proper dimer formation. This dimeric coiled coil sequence also contains a promiscuous HTL epitope

(SEQ ID NO: 10)
ARFVAALSMRLAE

that is within this coiled coil sequence predicted by NetMHCII to strongly bind to most of the major human MHC II molecules with the binding affinities as listed below.

TABLE 3
Predicted binding affinities of the HTL epitope
of SEQ ID NO: 10 to human MHCII molecules

| Haplotype | Binding affinity [nM] |
|---|---|
| DRB10101 | 4.7 |
| DRB10301 | 15.9 |
| DRB10401 | 8.0 |
| DRB10404 | 32.0 |
| DRB10405 | 28.0 |
| DRB10701 | 2.4 |
| DRB10802 | 192.4 |
| DRB10901 | 6.9 |
| DRB11101 | 23.8 |
| DRB11301 | 92.8 |
| DRB11501 | 6.2 |
| DRB30101 | 172.5 |
| DRB40101 | 93.2 |
| DRB50101 | 7.3 |

The P domain or, alternatively, only the P2 subdomain of any one of the norovirus strains is then attached to the dimeric coiled coil D2 by means of a flexible linker with residues GSGS. The particular norovirus sequence that was chosen is the norovirus Hu/1968/US (Jiang X et al., Virology 1993; 195(1):51-61) with the corresponding pdb-entry code 1IHM for the X-ray crystal structure. It contains residues 223 to 520 which are the P domain (lacking the 10 C-terminal residues 521-530 because these 10 residues are disordered in the X-ray crystal structure and because they are heavily positively charged) plus 3 amino acids of C-terminal end of the S domain according to the nomenclature presented by Prasad BVV et al., Science 1999; 286:287-290. The residue threonine 223 was carefully chosen by computer visualization programs to be the attachment point to the Noro-SAPN because it is the closest contact between the strands across the 2-fold axis in the crystal structure of the viral capsid.

This design then results in the following sequence that was used for protein expression, purification and biophysical analysis:

(SEQ ID NO: 1)
MGHHHHHHASGSWEKWNAKWDEWKNDWNDWRRDWQAWVDDWAYWTLTWK
YGELYSKLAELERRNEELERRLEELARFVAALSMRLAELERRNEELARG
SGSTVEQKTRPFTLPNLPLSSLSNSRAPLPISSMGISPDNVQSVQFQNG
RCTLDGRLVGTTPVSLSHVAKIRGTSNGTVINLTELDGTPFHPFEGPAP
IGFPDLGGCDWHINMTQFGHSSQTQYDVDTTPDTFVPHLGSIQANGIGS
GNYVGVLSWISPPSHPSGSQVDLWKIPNYGSSITEATHLAPSVYPPGFG
EVLVFFMSKMPGPGAYNLPCLLPQEYISHLASEQAPTVGEAALLHYVDP
DTGRNLGEFKAYPDGFLTCVPNGASSGPQQLPINGVFVFVSWVSRFYQL
KPVGTAS

### Examples

The following examples are useful to further explain the invention but in no way limit the scope of the invention.

### Cloning

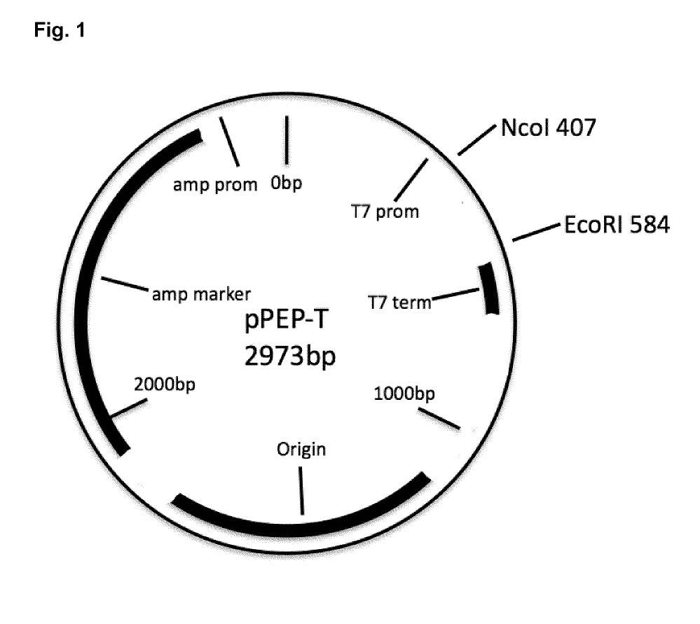

The DNA coding for the nanoparticle constructs were prepared using standard molecular biology procedures. Plasmids containing the protein sequence of SEQ ID NO:1 were constructed by cloning into the NcoI/EcoRI restriction sites of the basic SAPN expression construct of FIG. 1 to yield noro-SAPN.

### Expression

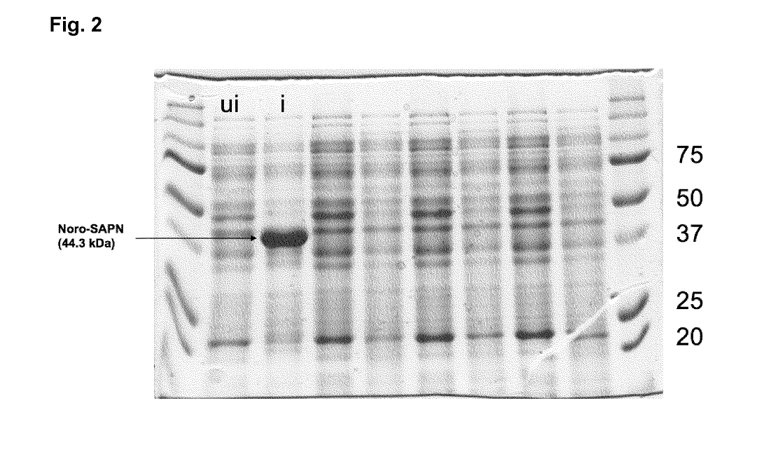

The plasmids were transformed into Escherichia coli BL21 (DE3) cells, which were grown in Luria broth with ampicillin at 37° C. Expression was induced with isopropyl βD-thiogalactopyranoside. Four hours after induction, cells were removed from 37° C. and harvested by centrifugation at 4,000×g for 15 min. The cell pellet was stored at −20° C. The pellet was thawed on ice and suspended in a lysis buffer consisting of 9 M urea, 100 mM NaH₂PO₄, 10 mM Tris pH 8, 20 mM imidazole, and 0.2 mM Tris-2-carboxyethyl phosphine (TCEP). The protein expression level was assessed by sodium dodecyl sulfate polyacrylamide gel electrophoresis (SDS-PAGE) and is shown in FIG. 2.

### Purification

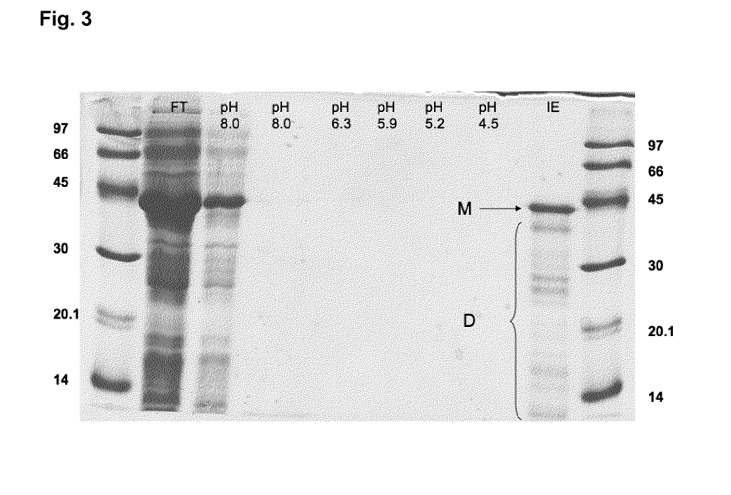

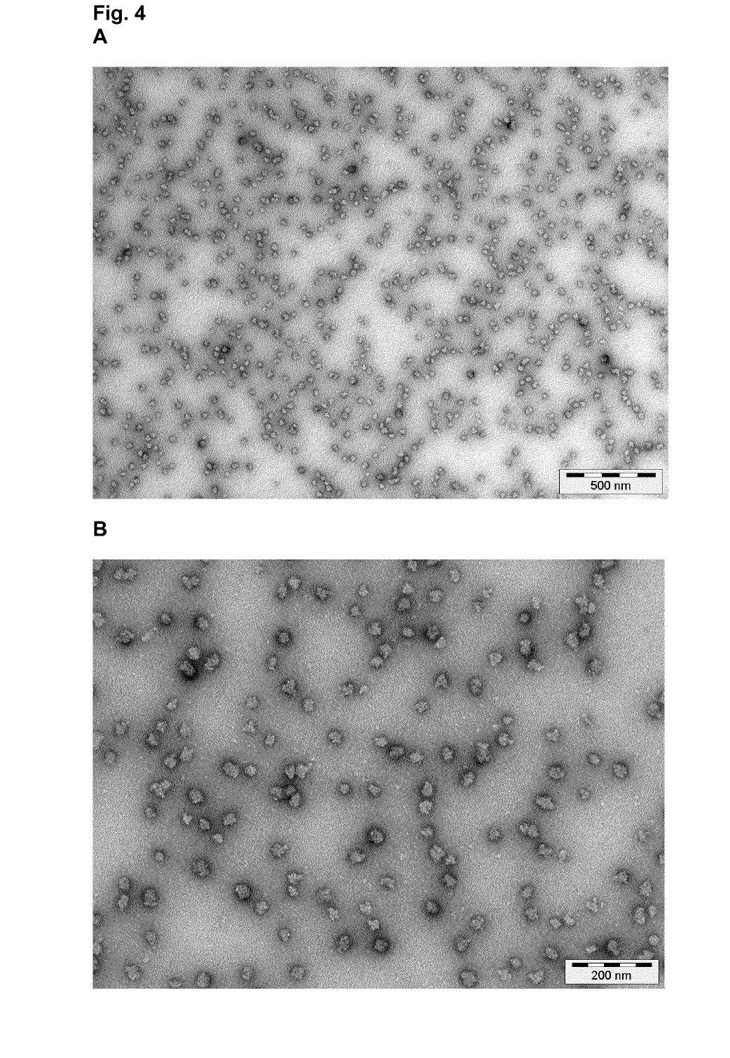

Cells were lysed by sonication and the lysate was cleared by centrifuging at 30,500×g for 45 min. The cleared lysate was incubated with Ni-NTA Agarose Beads (Qiagen, Valencia, Calif., USA) for at least 1 hour. The column was washed with lysis buffer and then a buffer containing 9 M urea, 500 mM NaH₂Pa₄, 10 mM tris pH 8, 20 mM imidazole, and 0.2 mM TCEP. Protein was eluted with a pH gradient: 9 M urea, 100 mM NaH₂PO₄, 20 mM citrate, 20 mM imidazole, and 0.2 mM TCEP. Subsequent washes were done at pH 6.3, 5.9, 5.2 and 4.5. Following the pH gradient, a gradient of lysis buffer with increasing imidazole strength was used to further elute the protein. Purity was assessed by sodium dodecyl sulfate polyacrylamide gel electrophoresis (SDS-PAGE) as shown in FIG. 3.

### Refolding

Quick refolding of Noro-SAPN. For quick refolding, 4 ml of a solution with a concentration of 1.8 mg/ml was added to a buffer solution as indicated in Table 4, to a final concentration of 0.05 mg/ml. The solution was then concentrated to 0.3. mg/ml, then analyzed by electron microscopy (37′000×) in a dilution of 0.1 mg/ml.

TABLE 4
Buffers used for refolding of Noro-SAPN

| | | | | | | |
|---|---|---|---|---|---|---|
| | | NaCl | MES | HEPES | Glycerol | TCEP |
| No. | pH | (mM) | (mM) | (mM) | (%) | (mM) |

First round—screening

| | | | | | | |
|---|---|---|---|---|---|---|
| 1 | 6.5 | 50 | 20 | — | 5 | — |
| 2 | 6.5 | 150 | 20 | — | 5 | — |
| 3 | 7.5 | 50 | — | 20 | 5 | — |
| 4 | 7.5 | 150 | — | 20 | 5 | — |

Second round—refinement

| | | | | | | |
|---|---|---|---|---|---|---|
| 5 | 6.8 | 25 | 20 | — | 5 | — |
| 6 | 6.8 | 50 | 20 | — | 5 | — |
| 7 | 6.8 | 80 | 20 | — | 5 | — |
| 8 | 6.8 | 25 | 20 | — | 5 | 5 |
| 9 | 6.8 | 50 | 20 | — | 5 | 5 |
| 10 | 6.8 | 80 | 20 | — | 5 | 5 |

MES = 2-Morpholinoethanesulfonic acid
HEPES = 2-[4-(2-Hydroxyethyl)piperazin-1-yl]ethanesulfonic acid
TCEP = Tris-2-carboxyethyl phosphine

Results of electron microscopy of the second round probes 5 to 10:

5) The particles vary in size. Background of unfolded protein (or very small particles) is high, particle mean size is 32 nm.

6) The particles look nice, not completely homogenous in size, low background. The particle mean size is 32 nm.

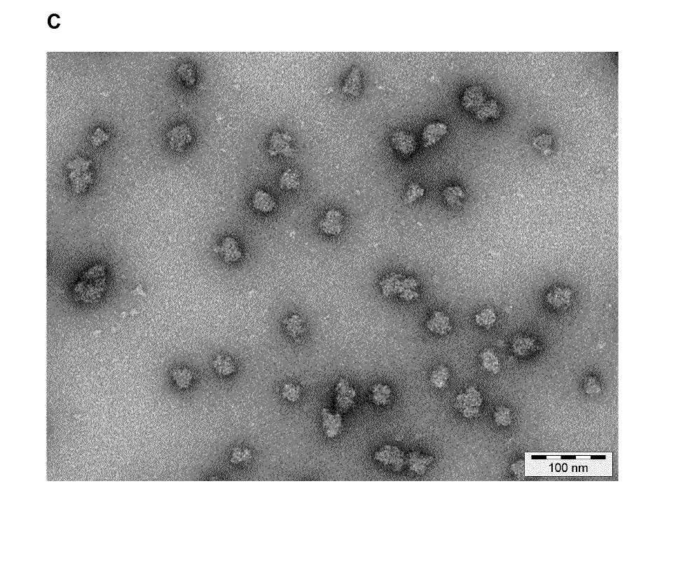

7) The particles look nice, slightly bigger in diameter compared to lower salt concentration (35 nm). Low background, concentration of the particles looks higher compared to probe 6 with 50 mM NaCl. See FIG. 4.

Particles refolded under reducing conditions (8, 9, 10, addition of 5 mM TCEP) look less round in shape, more aggregation and background was observed compared to non-reduced conditions.

1. A self-assembling peptide nanoparticle consisting of aggregates of a multitude of building blocks of formula (I) consisting of a continuous chain comprising a peptide oligomerization domain D1, a linker segment L, a peptide oligomerization domain D2, and the P domain or the P2 subdomain of norovirus protein VP1
D1-L-D2-P (I),
wherein D1 is a coiled coil peptide that forms oligomers (D1)ₘ of m subunits D1, D2 is a coiled coil peptide that forms dimers (D2)₂ of 2 subunits D2, m is either 3 or 5, L is a bond or a short linker segment, either D1 or D2 or both D1 and D2 incorporate one or more T-cell epitopes within the oligomerization domain, and wherein D2 is substituted by P representing the P domain or the P2 subdomain of the norovirus VP1 protein.

2. The peptide nanoparticle according to claim 1 wherein the substituent P is the P domain of the norovirus VP1 protein.

3. The peptide nanoparticle according to claim 1 wherein the substituent P is the P2 subdomain of the norovirus VP1 protein.

4. The peptide nanoparticle according to claim 1 wherein the oligomerization domain D1 is the pentamerization domain of the tryptophane zipper or a derivative thereof

5. The peptide nanoparticle according to claim 1 wherein at least one of the epitopes is a HTL epitope.

6. The peptide nanoparticle according to claim 1 wherein the sequence D1-L-D2-P comprises a series of optionally overlapping T-cell epitopes.

7. A pharmaceutical composition comprising a peptide nanoparticle according to claim 1.

8. A method of vaccinating a human or non-human animal, which comprises administering an effective amount of a peptide nanoparticle according to claim 1 to a subject in need of such vaccination.

9. A monomeric building block of formula (I) consisting of a continuous chain comprising a peptide oligomerization domain D1, a linker segment L, a peptide oligomerization domain D2, and the P domain or the P2 subdomain of norovirus protein VP1
D1-L-D2-P (I),
wherein D1 is a coiled coil peptide that forms oligomers (D1)ₘ of m subunits D1, D2 is a coiled coil peptide that forms dimers (D2)₂ of 2 subunits D2, m is either 3 or 5, L is a bond or a short linker segment, either D1 or D2 or both D1 and D2 incorporate one or more T-cell epitopes within the oligomerization domain, and wherein D2 is substituted by P representing the P domain or the P2 subdomain of the norovirus VP1 protein.

10. The monomeric building block of claim 9 which is the peptide of SEQ ID NO:1

11. The monomeric building block according to claim 9 wherein the substituent P is the P domain of the norovirus VP1 protein.

12. The monomeric building block according to claim 9 wherein the substituent P is the P2 subdomain of the norovirus VP1 protein.

13. The monomeric building block according to claim 9 wherein the oligomerization domain D1 is the pentamerization domain of the tryptophane zipper or a derivative thereof

14. The monomeric building block according to claim 9 wherein at least one of the epitopes is a HTL epitope.

15. The monomeric building block according to claim 9 wherein the sequence D1-L-D2-P comprises a series of optionally overlapping T-cell epitopes.


SEQUENCE LISTING (10 sequences)

SEQ ID NO: 1 - protein, 399 aa, Artificial Sequence
  Synthetic Construct
MGHHHHHHASGSWEKWNAKWDEWKNDWNDWRRDWQAWVDDWAYWTLTWKYGELYSKLAELERRNEELERRLEELARFVAALSMRLAELERRNEELARGSGSTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTAS

SEQ ID NO: 2 - protein, 11 aa, Artificial Sequence
  Synthetic Construct
KLKLLLLLKLK

SEQ ID NO: 3 - protein, 12 aa, Artificial Sequence
  Synthetic Construct
MGHHHHHHASGS

SEQ ID NO: 4 - protein, 53 aa, Artificial Sequence
  Synthetic Construct
SSNAKWDQWSSDWQTWNAKWDQWSNDWNAWRSDWQAWKDDWARWNQRWDNWAT

SEQ ID NO: 5 - protein, 30 aa, Artificial Sequence
  Synthetic Construct
WEKWNAKWDEWKNDWNDWRRDWQAWVDDWA

SEQ ID NO: 6 - protein, 16 aa, Artificial Sequence
  Synthetic Construct
YQTLTEKYGEKYSKMA

SEQ ID NO: 7 - protein, 37 aa, Artificial Sequence
  Synthetic Construct
WEKWNAKWDEWKNDWNDWRRDWQAWVDDWAYWTLTWK

SEQ ID NO: 8 - protein, 46 aa, Artificial Sequence
  Synthetic Construct
ELYSKLAELERRNEELERRLEELARFVAALSMRLAELERRNEELAR

SEQ ID NO: 9 - protein, 46 aa, Artificial Sequence
  Synthetic Construct
ELYSKLAELERRLEELERRLEELARFVAALSMRLAELERRLEELAR

SEQ ID NO: 10 - protein, 13 aa, Artificial Sequence
  Synthetic Construct
ARFVAALSMRLAE